SEQUENCE LISTING (30 sequences)

SEQ ID NO: 1 - protein, 13 aa, Homo sapiens
CASSAGLLGEQYF

SEQ ID NO: 2 - DNA, 39 bp, Homo sapiens
tgtgccagcagtgcggggcttttgggggagcagtacttc

SEQ ID NO: 3 - protein, 13 aa, Homo sapiens
CAIQMYPAYEQYF

SEQ ID NO: 4 - DNA, 39 bp, Homo sapiens
tgtgccattcagatgtacccagcctacgagcagtacttc

SEQ ID NO: 5 - protein, 16 aa, Homo sapiens
CASSYSRGQGSYEQFF

SEQ ID NO: 6 - DNA, 48 bp, Homo sapiens
tgtgccagcagttactcaaggggacaggggtcctatgagcagttcttc

SEQ ID NO: 7 - protein, 13 aa, Homo sapiens
CASRPGQKSMSSS

SEQ ID NO: 8 - DNA, 41 bp, Homo sapiens
tgtgccagcagaccgggacagaaatcaatgagcagttcttc

SEQ ID NO: 9 - protein, 18 aa, Homo sapiens
CASSYSGVLATVDTGELF

SEQ ID NO: 10 - DNA, 55 bp, Homo sapiens
tgtgccagcagttactcgggggtccttgcgaccgtggacaccggggagctgtttt

SEQ ID NO: 11 - protein, 14 aa, Homo sapiens
CASSKARTNKQAVL

SEQ ID NO: 12 - DNA, 46 bp, Homo sapiens
tgtgccagcagcaaagcaaggaccaataaacaatgagcagttcttc

SEQ ID NO: 13 - protein, 13 aa, Homo sapiens
CSARALLSTEAFF

SEQ ID NO: 14 - DNA, 39 bp, Homo sapiens
tgcagtgctagagctttattgagcactgaagctttcttt

SEQ ID NO: 15 - protein, 15 aa, Homo sapiens
CASSLDSSSYNEQFF

SEQ ID NO: 16 - DNA, 45 bp, Homo sapiens
tgtgctagcagcttggactcgagttcttacaatgagcagttcttc

SEQ ID NO: 17 - protein, 14 aa, Homo sapiens
CSVEGPTLYNEQFF

SEQ ID NO: 18 - DNA, 42 bp, Homo sapiens
tgcagcgttgaagggccaacgttgtacaatgagcagttcttc

SEQ ID NO: 19 - protein, 15 aa, Homo sapiens
CASSRPGAGTDTQYF

SEQ ID NO: 20 - protein, 45 aa, Homo sapiens
TGTGCCAGCAGCCGACCGGGAGCGGGCACAGATACGCAGTATTTT

SEQ ID NO: 21 - protein, 16 aa, Homo sapiens
CASRSLTGGGAETQYF

SEQ ID NO: 22 - DNA, 48 bp, Homo sapiens
tgtgccagcaggagcctcactggtgggggggcggagacccagtacttc

SEQ ID NO: 23 - protein, 13 aa, Homo sapiens
CSASASYNSPLHF

SEQ ID NO: 24 - DNA, 39 bp, Homo sapiens
tgcagtgctagcgccagttataattcacccctccacttt

SEQ ID NO: 25 - protein, 13 aa, Homo sapiens
CSAREGGTPGSCF

SEQ ID NO: 26 - DNA, 41 bp, Homo sapiens
tgcagtgctagagaggggggaacaccggggagctgtttttt

SEQ ID NO: 27 - protein, 15 aa, Homo sapiens
CSVEEWASRYNEQFF

SEQ ID NO: 28 - DNA, 45 bp, Homo sapiens
tgcagcgttgaagagtgggctagcagatacaatgagcagttcttc

SEQ ID NO: 29 - protein, 14 aa, Homo sapiens
CASTVDSLDTEAFF

SEQ ID NO: 30 - DNA, 42 bp, Homo sapiens
tgtgccagcaccgtggacagtctggacactgaagctttcttt

(19) United States
(12) Patent Application Publication
(10) Pub. No.: US 2014/0065629 A1
(43) Pub. Date: Mar. 6, 2014

(54) METHODS OF TREATING DISEASES

(71) Applicants: ISRAEL BARKEN, San Diego, CA (US); Derren Barken, San Diego, CA (US)

(72) Inventors: ISRAEL BARKEN, San Diego, CA (US); Derren Barken, San Diego, CA (US)

(21) Appl. No.: 13/970,537

(22) Filed: Aug. 19, 2013

Related U.S. Application Data
(63) Continuation-in-part of application No. PCT/US2012/052944, filed on Aug. 29, 2012.

(51) Int. Cl. C12Q 1/68

(52) U.S. Cl. CPC C12Q 1/6869
USPC 435/6.12; 435/6.1

(57) ABSTRACT

In one example, the present invention comprises deliberate tumor insult and sequencing of the T cell repertoire before and after the insult in order to detect and sequence the TCR alpha and beta loci of highly expanded T cell clonotypes. In some examples, this information is used in turn to create autologous genetically engineered T cells with TCR sequences that target the individual's tumor.

### CROSS-REFERENCE TO RELATED APPLICATIONS

This application claims priority to U.S. Provisional Application Ser. No. 61/528,657, filed Aug. 29, 2011, which is incorporated in its entirety herein.

### BACKGROUND OF THE INVENTION

The field of the present invention relates generally to treatments for cancer.

Cancer is a large, heterogeneous class of diseases in which a group of cells display uncontrolled growth, invasion that intrudes upon and destroys adjacent tissues, and often metastasizes, wherein the tumor cells spread to other locations in the body via the lymphatic system or through the bloodstream. These three malignant properties of cancer differentiate malignant tumors from benign tumors, which do not grow uncontrollably, directly invade locally, or metastasize to regional lymph nodes or distant body sites like brain, bone, liver, or other organs.

Cancer is one of the leading causes of death in developed countries; therefore there is a need for improved methods for treating cancer.

### SUMMARY OF THE INVENTION

One embodiment provides a method for identifying DNA or RNA sequences of lymphocyte receptors that are present in greater numbers of lymphocytes after a medical procedure comprising drawing pre-procedure blood from a cancer patient, carrying out a medical procedure on the patient, drawing blood from the patient at one or more times following the medical procedure, purifying lymphocytes from any one or more of the pre-procedure blood draw(s), and/or the post-procedure blood draw(s); isolating DNA, amplifying (if necessary) and sequencing the DNA, and identifying lymphocytes and/or lymphocyte receptor sequences that have expanded following the procedure.

Another embodiment provides a method wherein the patient is selected based on the severity of cancer (Gleason Score for example) and/or patient treatment status.

Another embodiment provides a method wherein the procedure comprises one or more surgical procedure or procedure, nonsurgical procedure or procedure, or exposure to a drug.

Another embodiment provides a method wherein the cancer is prostate cancer and the medical procedure comprises one or more of cryosurgery, radical prostatectomy, prostate biopsy, radiation therapy, brachytherapy, CyberKnife™ procedures, electroporation, high frequency ultrasound (HIFU), photodynamic therapy, prostate laser surgery, androgen deprivation therapy, and chemotherapy.

Another embodiment provides a method wherein the procedure leads to an immunogenic response.

Another embodiment provides a method wherein the procedure results in a change in the population lymphocytes.

Another embodiment provides a method wherein the lymphocytes include T-cells displaying various TCRs.

Another embodiment provides a method wherein treatment of cancer comprises inducing in a patient an immunologic response incorporating clonotypes identified by the method of claim 1.

Another embodiment provides a method wherein the method further comprises selecting clonotypes as highly expanded if their frequency (in the measured repertoire) is 0.5% or greater.

Another embodiment provides a method wherein the method further comprises selecting a clonotype that is absent or not highly expanded prior to cryosurgery, but which is highly expanded after cryosurgery as a tumor associated clonotype.

Another embodiment provides a method wherein the method further comprises selecting a clonotype as a tumor specific clonotype if it is highly expanded both before and after a medical procedure, but has a frequency that increases from before to after the procedure, wherein the increase is statistically significant using an appropriate multiple hypothesis testing statistical method to stringently limit the false discovery rate.

Another embodiment provides a method wherein the method further comprises extracting tissue from the patient for use in an in-vitro assay of autologous engineered T cells.

Another embodiment provides a method wherein the procedure comprises receptor chain pairing.

Another embodiment provides a method wherein chain pairing is carried out in silico by computer methods.

Another embodiment provides a method wherein chain pairing involves immunology gene alignment software.

Another embodiment provides a method wherein the software is selected from IMGT, JOINSOLVER, VDJSolver, SoDA, iHMMune-align, or other similar tools.

Another embodiment provides a method wherein chain pairing involves using VDJ antibodies.

Another embodiment provides a method wherein the method further comprises obtaining antibodies for the identified segments and use the antibodies to purify a subset of cells which express that gene segment in their (surface) receptors (e.g. using FACS, or immunomagnetic selection with microbeads).

Another embodiment provides a method wherein the method further comprises sequencing a subset of cells which have been purified for the desired gene segments.

Another embodiment provides a method wherein chain pairing is carried out using multi-well sequencing or single cell sequencing.

Another embodiment provides a method wherein the method further comprises genetic engineering of autologous T-cells, acquired by leukapheresis, to display the TCR or CAR of the induced clonotype(s).

Another embodiment provides a method wherein a T-cell is engineered to display a functional TCR.

Another embodiment provides a method wherein a chimeric cell is engineered in which a T-cell displays an alternative type of receptor such as a chimeric antigen receptor.

Another embodiment provides a method wherein the method further comprises an in-vitro assay.

Another embodiment provides a method wherein the engineered T-cells are incubated with tumor tissue or lysate.

Another embodiment provides a method wherein one or more effects are measured during the incubation such as cytokine concentration, cell proliferation, and the like. In some embodiments, the effects of various adjuvants are quantified.

Another embodiment provides a method wherein the engineered T cells are provided as a treatment for cancer.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a flowchart corresponding to protocol for the study of in-vitro and in vivo efficacy of autologous engineered T cells.

FIG. 2 shows excerpts from a sample report provided by a TCR profiling vendor (Evrotec).

FIG. 3 shows excerpts from a sample report provided by a TCR profiling vendor (Evrotec).

FIG. 4 shows a scatterplot demonstrating the reproducibility of the method, provided by a TCR and BCR profiling vendor (Adaptive TCR)

### DETAILED DESCRIPTION OF THE INVENTION

In one example, the present invention comprises a medical procedure, and sequencing of the T cell repertoire before and after a medical procedure, in order to detect and sequence the TCR alpha and beta loci of highly expanded T cell clonotypes. In some examples, this information is used in turn to create autologous genetically engineered T cells with lymphocyte receptors that target the individual's tumor.

According to a seminal paper (Hanahan D, Weinberg R A (January 2000). “The Hallmarks of Cancer”. Cell 100 (1): 57-70), all cancers share six hallmarks. In addition, cancers generally evade immune destruction. The field of cancer immunology, which studies this hallmark of cancer, encompasses the topics of immunosurveillance, immunoediting, immune tolerance, immune escape, and immunotherapy.

Designed immunotherapies have only recently been approved for oncological indications. However, the history of oncology includes interventions which, although designed primarily to destroy cancerous tissue, have in some rare cases had unforeseen, fortuitous, and systemic secondary effects. Specifically, there are rare clinical reports of cryosurgical interventions which, in addition to destroying the targeted cancerous tissue, also resulted in the regression of secondary, metastatic cancerous tissue. It has long been hypothesized that this effect was immunologically mediated, and efforts have been made to enhance its effects using adjuvants.

Separately, the study of the T cell receptor repertoire is being transformed by the advent of high throughput sequencing technologies. Initially, the T cell repertoire was widely studied using techniques such as spectratyping. Recently, and particularly with increased read lengths which span more of the CDR3 region, there has been a renaissance in the sequencing of the T cell repertoire.

Finally, the tools of genetic engineering/gene therapy have steadily improved. Lymphocytes have been attractive targets for these new techniques, for a variety of reasons. The state of the art in engineering T cells to, for example, carry designed T cell receptors, has advanced dramatically. This has created new opportunities to design the engineered receptors rationally and intelligently.

Described herein are methods for combining a plurality of biological methodologies in new ways to improve the treatment of cancers. In some embodiments, the described methods of treatment incorporate a medical procedure which results in an in-situ insult to an individual's tumor (such as cryosurgery). In some embodiments, the methods described herein comprise techniques for analyzing an individual's repertoire of lymphocyte receptors. Also described herein are methods that involve extracting lymphocytes, manipulating them ex vivo for therapeutic purposes, and then returning those lymphocytes to the individual to induce a therapeutic result.

The present invention describes a technique by which a medical procedure (also termed a tumor insult) may be employed to elicit an immunological response. This response may be analyzed in detail by receptor repertoire sequencing of lymphocytes. The analysis may be expected to reveal the receptor sequences of lymphocyte clonotypes which are specific to the cancer. These sequences may be used to genetically engineer lymphocytes which have the same or similar tumor specificity but which may be manipulated ex vivo to enhance their anti-tumor efficacy when returned to the body as an immunotherapy.

In one aspect, the invention combines an in-situ insult to a tumor, a series of one or more measurements of the T and/or B cell receptor repertoire, an analysis of the T and/or B cell repertoire measurements in order to identify tumor specific T and/or B cell receptor sequences expressed in tumor specific T and/or B cell clonotypes, and T and/or B cell gene therapy techniques to employ the identified tumor specific TCR and/or BCR sequences for therapeutic purposes.

In one aspect, the invention provides methods for generating an insult to a tumor which has the effect of provoking an immune response which may be measured.

In one aspect, the invention provides methods for measuring said immune response in such a way as to be useful for the design of a gene therapy which is efficacious against a patient's tumor.

In one aspect, the invention provides methods for generating a gene therapy and/or immunotherapy which utilizes the information which is made available by an analysis of sequencing data sets which describe a T cell receptor repertoire.

In one aspect, the invention provides a description of the overall design which combines the individual elements described above into a multi-step clinical strategy which is efficacious.

Aspects of the present invention may be better understood in reference to the Figures.

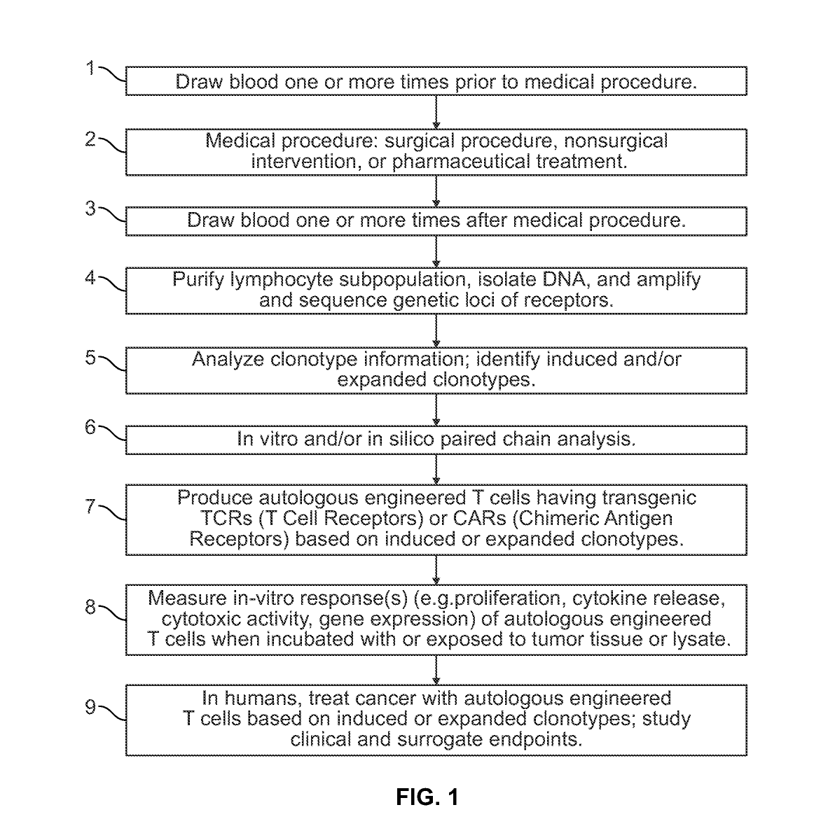

Referring to FIG. 1, this figure describes a method for determining T-cell receptors induced or expanded by tumor intervention. FIG. 1 describes an in-vitro (steps 1-8) and in-vivo (step 9) study. Patients may be selected for the study based on the severity of their cancer (Gleason Score for example), their hormonal treatment status (androgen deprivation therapy, for example), and the like. In some embodiments, a population of patients are selected for study that statistically represent the patient population as a whole and/or a subset of the patient population suitable for treatment using the methods described herein. In the first step, one or more pre-operative blood draws are taken from a patient with cancerous tissue. These blood draws may be analyzed immediately or preserved for later analysis using any suitable method.

In a second step, the cancerous tumor and/or cancerous tissue of the patient(s) are “insulted” or “intervened”, meaning that the tissue is acted upon, treated, surgically altered, altered by radiation or other nonsurgical intervention, or exposure to a drug and the like. Any medical procedure known in the art of cancer treatment is appropriate, and may include various types of prostate surgery in various embodiments. Non-limiting examples include one or more of cryosurgery, radical prostatectomy, prostate biopsy, radiation therapy, brachytherapy, CyberKnife™ procedures, electroporation, high frequency ultrasound (HIFU), photodynamic therapy, prostate laser surgery, androgen deprivation therapy, and chemotherapy. In some embodiments, a method of tumor insult is selected that is known to lead to an immunogenic response. Without being limited to any particular theory, it is hypothesized that intervention in the tumor will result in a change in the population lymphocytes. The lymphocytes include T-cells displaying various TCRs. In some embodiments, TCRs that are specific to the cancerous tumor can be utilized in methods for treatment of the cancer. In some embodiments, the cancerous tissue is analyzed immediately or preserved for later analysis. Methods of analysis can include methods described herein or any suitable method of genetic and/or biochemical analysis known to those skilled in the art. In other embodiments, the tissues are preserved, optionally in FFPE.

In a third step of the method depicted in FIG. 1, blood is drawn from the patient at various times following intervention of the cancerous tissue. Any time period may be suitable and may be adjusted to coincide with the timing of an immunological response in the patient. In some embodiments, blood is drawn a plurality of times. In some embodiments, blood may be drawn 1 day, 2 days, 3 days, 5 days, 7 days, 10 days, 14 days, 21 days, 30 days, and the like following surgery. The post-operative and/or post-intervention blood samples may be analyzed immediately or preserved for later analysis.

In Step 4 of FIG. 1, DNA is isolated from any one or more of the pre-operative blood draw(s), the cancerous tissue, and/or the post-operative blood draw(s). In some embodiments, DNA is extracted from a mixed tissue or mixed cell-type sample, optionally from whole blood or cancerous tissue. This embodiment may eliminate the need for certain sample processing steps, whereby the genetic loci of interest can be interrogated from a mixed DNA sample. In another embodiment, the blood and/or tissue samples are first enriched for certain lymphocytes, optionally by whole blood fractionation for CD8+ cells. Whether from an enriched sample, or from a non-enriched sample, DNA can be isolated according to any suitable method known to those skilled in the art. Ones skilled in the art will be aware that kits are commercially available that provide the necessary reagents and plastic-ware needed for DNA isolation, PCR amplification, DNA sequencing, and the like. Life Technologies is one commercial supplier of molecular biology kits.

In step 4 of FIG. 1, the extracted DNA is then amplified (if necessary) and sequenced. Specifically, the DNA encoding the lymphocyte receptors is amplified in some embodiments, optionally the T-cell receptors. T-cell receptors consist of alpha (α) and beta (β) chains. In some embodiments, both the alpha and beta chains of a TCR are amplified. In other embodiments, various loci can be amplified separately. For example, the alpha and beta chains of a TCR can be amplified separately to yield two PCR products. Skilled persons will be familiar with methods in polymerase chain reaction (“PCR”) that are suitable for DNA amplification including design of short, single stranded pieces of DNA that serve as PCR primers, adjustment of annealing, melting and extension times and temperatures and the like such that high quality PCR products are produced. Some DNA amplification procedures may be conducted in multi-well plates.

Step 4 of FIG. 1 includes DNA sequencing of the DNA. Methods of DNA sequencing are well known in the art and have improved rapidly in recent years in features such as read length, improved throughput, reduced cost and the like. One suitable method of DNA sequencing is pyrosequencing. Pyrosequencing is a method of DNA sequencing (determining the order of nucleotides in DNA) based on the “sequencing by synthesis” principle. It differs from Sanger sequencing, in that it relies on the detection of pyrophosphate release on nucleotide incorporation, rather than chain termination with dideoxynucleotides. DNA sequencing results in sequence data of adenine (A), cytosine (C), thymine (T) and guanine (G) that is analyzed by computer methods in the following step.

Step 5 includes methods for identifying lymphocytes and/or lymphocyte receptor sequences that have expanded following intervention of the cancerous tissue. By “expansion” it is meant that the number of members of a clonotype is greater following intervention, than before intervention. Expansion can be quantified by comparing the amount of amplified DNA for a given alpha and/or beta chain from samples before and after intervention. The methods of Step 5 are often computer-based. In some embodiments, clonotypes are considered highly expanded if their frequency (in the measured repertoire) is 0.5% or greater. In some embodiments, a clonotype which is absent or not highly expanded prior to cryosurgery or other medical procedure, but which is highly expanded after cryosurgery or other medical procedure, is inferred to be a tumor associated clonotype. In some embodiments, a clonotype will be inferred to be a tumor specific clonotype if it is highly expanded both before and after tumor intervention, but has a frequency that increases from before to after cryosurgery or other medical procedure, where the increase is statistically significant using an appropriate multiple hypothesis testing statistical method to stringently limit the false discovery rate.

It is understood that FIG. 1 is a depiction of generalized procedures and not limiting. For example, Step 4 of FIG. 1 does not limit the method to performing DNA extraction, amplification and sequencing simultaneously. Furthermore, there may be other steps not necessarily depicted in the figures, such as sample processing and the like. The present invention is only limited by the claims.

In some embodiments, Step 5 of FIG. 1 results in separate data comprising alpha chains that are induced upon intervention of the tumor and beta chains that are induced upon intervention of the tumor. Step 6 of the procedure depicted in FIG. 1 involves “paired chain analysis”. In this step, various methods can be utilized to pair induced alpha and beta chains such that the pairing results in a TCR that binds to an epitope of the cancerous tissue or otherwise leads to an immune response targeting the cancerous tissue. In some embodiments post-sequencing pairing may be unnecessary or relatively simple, for example in embodiments in which the alpha and beta chain pairing information is not lost in the procedure, such as if one were to sequence from single cells.

In some embodiments, the chain pairing may be assisted in silico by computer methods. For example specialized, publicly available immunology gene alignment software is available from IMGT, JOINSOLVER, VDJSolver, SoDA, iHMMune-align, or other similar tools for annotating VDJ gene segments.

In some embodiments, the chain pairing may be done using VDJ antibodies. For example, one may obtain antibodies for the identified segments and use the antibodies to purify a subset of cells which express that gene segment in their (surface) receptors (e.g. using FACS, or immunomagnetic selection with microbeads). One may then sequence from this subset of cells which have been purified for the desired gene segments. If necessary, this secondary sequencing may be done more deeply (i.e. at a higher resolution) than the first round of sequencing. In this second sequence data set, there will be far fewer induced clonotypes, greatly easing the task of chain pairing. Depending on the gene segments, there may be only one induced alpha chain and one induced beta chain for example.

In some embodiments, the chain pairing may be done by trial and error.

In some embodiments, the chain pairing may be done using multiwall sequencing. For example one may isolate gene segment purified cells or unpurified cells into a microwell plate, where each microwell has a very low number of cells. One can amplify and sequence the cells in each well individually, which provides another means to pair the chains of interest by sequencing on a single cell basis, facilitating the pairing of induced alpha and beta chains.

Step 7 of FIG. 1 includes genetic engineering of autologous T-cells to display the TCR or chimeric antibody receptor corresponding to the induced clonotype(s). Methods of genetic engineering are generally known in the art and can be found in (Sambrook et al. (2001) in “Molecular Cloning. A Laboratory Manual”, Cold Spring Harbor Press, Plainview, N.Y.) for example. The alpha and beta chains of the T-cells of this invention may be expressed independently in different hosts or in the same host. Preferably the alpha and beta chains are introduced into the same host to allow for formation of a functional T-cell receptor in the host cell. In some embodiments, the host cell is capable of inducing an immune response in a patient. The means by which the vector carrying the gene may be introduced into the cell include, but are not limited to, microinjection, electroporation, transduction, retroviral transduction or transfection using DEAE-dextran, lipofection, calcium phosphate, particle bombardment mediated gene transfer or direct injection of nucleic acid sequences encoding the T-cell receptors of this invention or other procedures known to one skilled in the art (Sambrook et al. (2001) in “Molecular Cloning. A Laboratory Manual”, Cold Spring Harbor Press, Plainview, N.Y.).

In some embodiments, a T-cell is engineered to display a functional TCR. In some embodiments, a chimeric cell may be engineered in which a T-cell displays an alternative type of receptor such as a B-cell receptor.

Step 8 of FIG. 1 includes in-vitro assays. In some embodiments, the autologous engineered T-cells from Step 7 of FIG. 1 are incubated with tumor tissue or lysate. In various embodiments, various effects are measured during the incubation such as cytokine concentration, cell proliferation, and the like. In some embodiments, the effects of various adjuvants are quantified.

Turning now to step 9 of FIG. 1, depicted herein is an exemplary sequence of steps which comprise a therapeutic strategy.

In step 9, autologous engineered tumor specific cells are returned to the patient's body in a manner suitable for treatment of the patient's cancer. In some embodiments, the cells further include one or more adjuvants suitable to illicit or amplify an immune response.

In step 9, the effects of the treatment are evaluated by reference to clinical and surrogate endpoints. The clinical endpoints may include one or more of overall survival, progression free survival, or tumor regression. The surrogate endpoints may include longitudinal measurements of cancer biomarkers. In the case of prostate cancer, available surrogate endpoints would include PSA (prostate specific antigen) and circulating tumor cells.

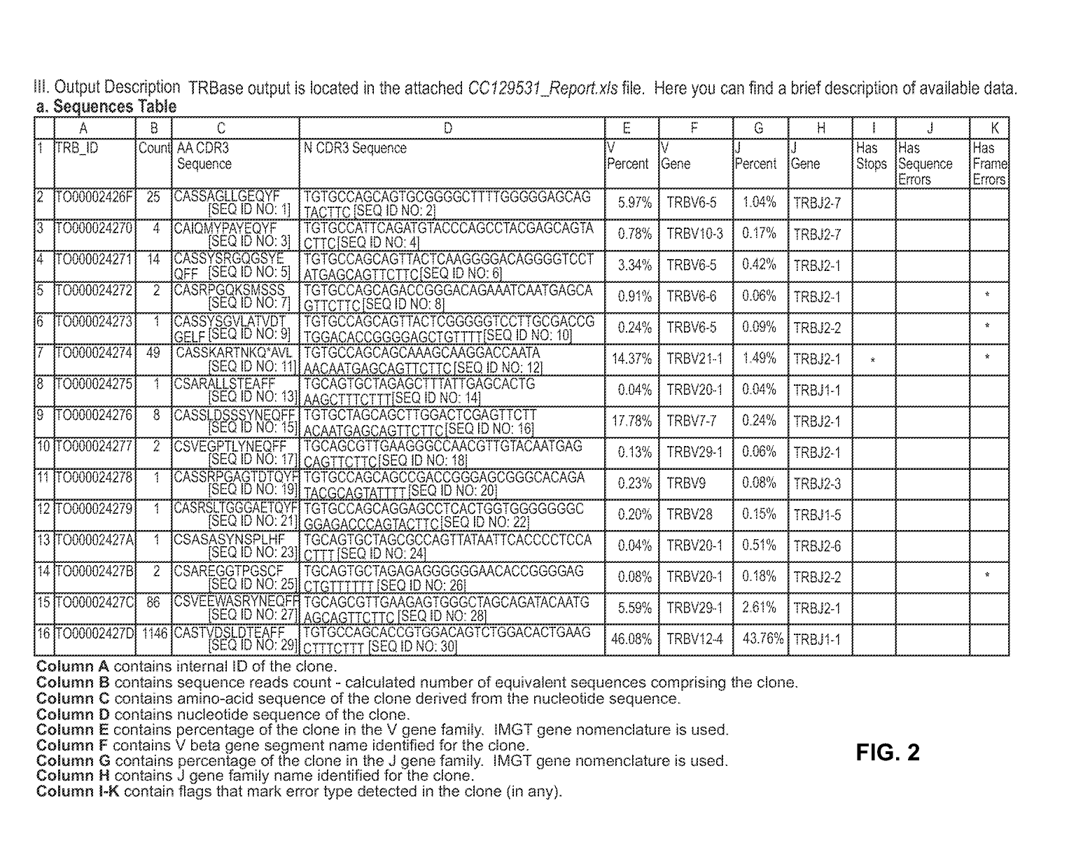

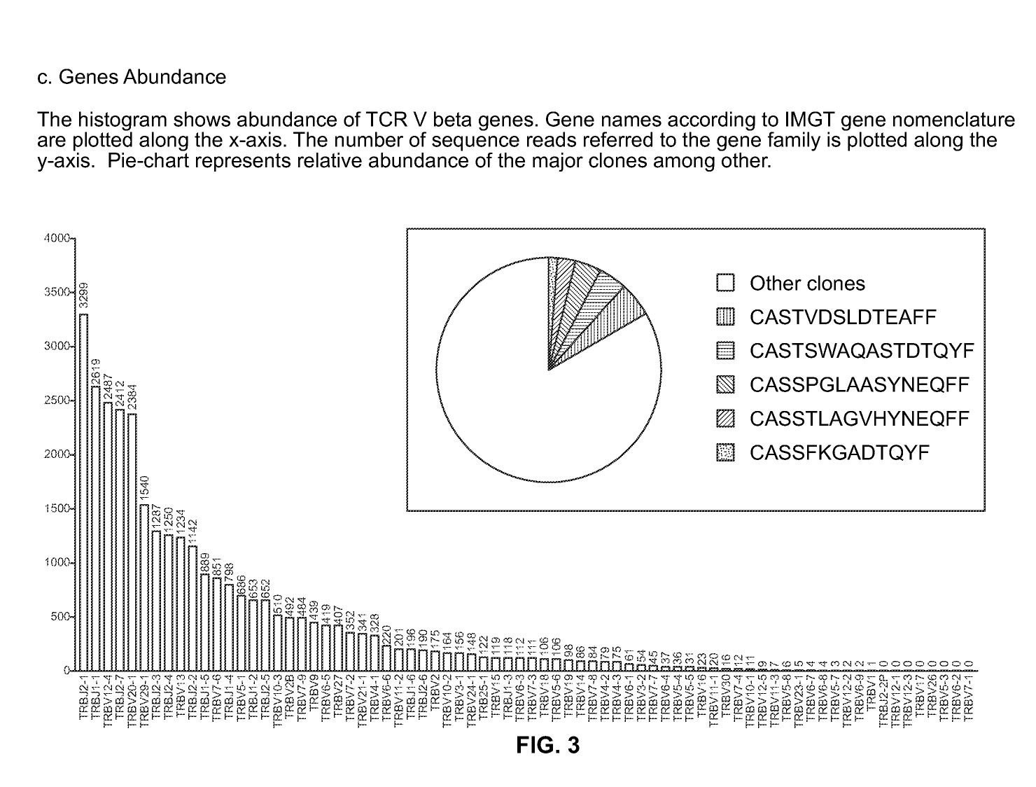

FIGS. 2 and 3 show that the TCR beta chain CDR3 (complementarity determining region 3) sequences and clonotype frequencies are commercially available. In these examples, the commercial provider is Evrotec.

FIG. 2 is an exemplary report showing among other things, a listing of clones, their sequence and read count, the percentage of the clone in the V gene family, the percentage of the clone in the J gene family.

FIG. 3 the abundance of T-cell receptor V beta genes depicted as a histogram and a pie chart. FIGS. 2 and 3 are examples of computer-based methods that can be used to identify T-cell receptor segments that increase in abundance following insulting the tumor.

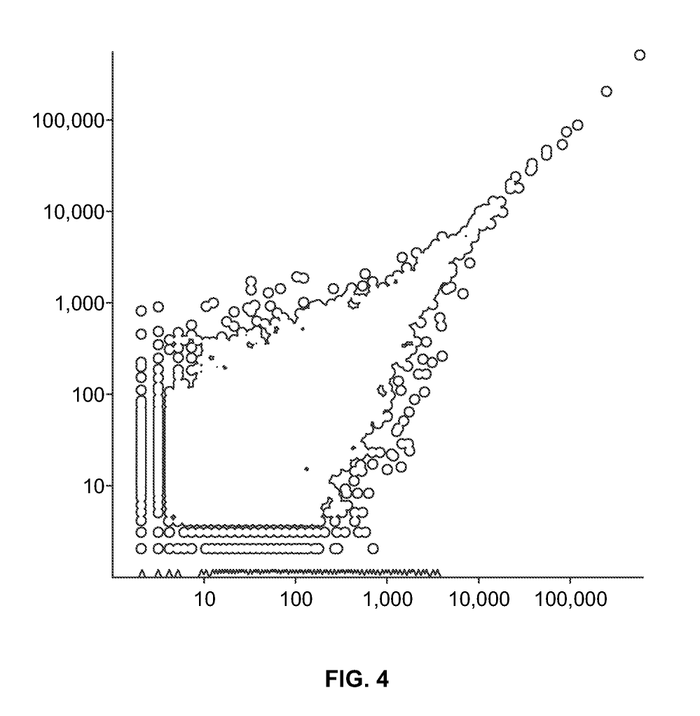

FIG. 4 shows a scatterplot demonstrating the reproducibility of the method. In this case, both the x-axis and y-axis are the same logarithmic scale plotting the same data, so perfectly reproducible data would fall on a 45 degree line ascending from the bottom left to the top right of the graph. FIG. 4 shows that a majority of the data points follow this 45 degree trend of reproducibility.

The methods disclosed herein can be used to treat and/or diagnose all types of cancer including but not limited to breast cancer, colon cancer, liver cancer and the like.

Either the primary or secondary tumors can be insulted.

In some embodiments, lymph material is removed or drawn from the patient in lieu of blood.

### DEFINITIONS

All technical terms have the standard accepted meaning in the art to which the present disclosure applies. Certain definitions may be found in U.S. Pat. No. 5,830,755, which is incorporated herein for the purpose of supplying definitions.

“Cryotherapy” is the local or general use of low temperatures in medical therapy or the removal of heat from a body part. “Cryoablation” is a process that uses extreme cold (cryo) to destroy and/or remove tissue (ablation). In cryoablation, the destroyed tissue is not necessarily removed from the body—the ablation may refer to removal from the tumor, but the destroyed tissue may remain in the body, enhancing the immune response.

“T lymphocytes” or “T cells” belong to a group of white blood cells known as lymphocytes, and play a central role in cell-mediated immunity. They can be distinguished from other lymphocyte types, such as B cells and natural killer cells (NK cells) by the presence of a special receptor on their cell surface called T cell receptors (TCR).

A “cluster of differentiation” (often abbreviated as CD) is a protocol used for the identification and investigation of cell surface molecules present on white blood cells, providing targets for immunophenotyping of cells. Physiologically, CD molecules can act in numerous ways, often acting as receptors or ligands (the molecule that activates a receptor) important to the cell. A signal cascade is usually initiated, altering the behavior of the cell (see cell signaling). Some CD proteins do not play a role in cell signaling, but have other functions, such as cell adhesion. CD for humans is numbered up to 350 most recently (as of 2009).

“CD8” (cluster of differentiation 8) is a transmembrane glycoprotein that serves as a co-receptor for the T cell receptor (TCR). Like the TCR, CD8 binds to a major histocompatibility complex (MHC) molecule, but is specific for the class I MHC protein. There are two isoforms of the protein, alpha and beta, each encoded by a different gene. In humans, both genes are located on chromosome 2 in position 2p12.

“T cell receptor” or “TCR” is a molecule found on the surface of T lymphocytes (or T cells) that is, in general, responsible for recognizing antigens bound to major histocompatibility complex (MHC) molecules. The binding between TCR and antigen is of relatively low affinity and is degenerate: that is, many TCR recognize the same antigen and many antigens are recognized by the same TCR. The TCR is composed of two different protein chains (that is, it is a heterodimer). In 95% of T cells in peripheral blood, this consists of an alpha (α) and beta (β) chain, whereas in 5% of T cells in peripheral blood, this consists of gamma and delta (γ/δ) chains. These percentages are different in other locations in the body.

“VDJ recombination” is also known as somatic recombination, is a mechanism of genetic recombination in the early stages of immunoglobulin (Ig) and T cell receptors (TCR) production of the immune system. V(D)J recombination nearly-randomly combines Variable, Diverse, and Joining gene segments of vertebrates, and because of its randomness in choosing different genes, is able to diversely encode proteins to match antigens from bacteria, viruses, parasites, dysfunctional cells such as tumor cells, and pollens. V(D)J recombination,

The “Gleason Grading system” is used to help evaluate the prognosis of men with prostate cancer. Together with other parameters, it is incorporated into a strategy of prostate cancer staging which predicts prognosis and helps guide therapy. A Gleason score is given to prostate cancer based upon its microscopic appearance. Cancers with a higher Gleason score are more aggressive and have a worse prognosis.

The “CyberKnife™” is a frameless robotic radiosurgery system used for treating benign tumors, malignant tumors and other medical conditions. The two main elements of the CyberKnife™ are (1) the radiation produced from a small linear particle accelerator and (2) a robotic arm which allows the energy to be directed at any part of the body from any direction. The CyberKnife™ system is a method of delivering radiotherapy, with the intention of targeting treatment more accurately than standard radiotherapy.

“High-Intensity Focused Ultrasound” (HIFU) or “high frequency ultrasound” is a highly precise medical procedure using high-intensity focused ultrasound to heat and destroy pathogenic tissue rapidly. It is one modality of therapeutic ultrasound, and, although it induces hyperthermia, it should not be confused with this technique, which heats much less rapidly and to much lower therapeutic temperatures (in general <45° C.).

A “biopsy” is a medical test involving the removal of cells or tissues for examination. It is the medical removal of tissue from a living subject to determine the presence or extent of a disease. The tissue is generally examined under a microscope by a pathologist, and can also be analyzed chemically. When an entire lump or suspicious area is removed, the procedure is called an excisional biopsy. When only a sample of tissue is removed with preservation of the histological architecture of the tissue's cells, the procedure is called an incisional biopsy or core biopsy. When a sample of tissue or fluid is removed with a needle in such a way that cells are removed without preserving the histological architecture of the tissue cells, the procedure is called a needle aspiration biopsy.

A “radical prostatectomy” is the surgical removal of all or part of a prostate gland in order to remove prostate cancer.

“Radiation therapy”, “radiation oncology”, or “radiotherapy” sometimes abbreviated to XRT, is the medical use of ionizing radiation, generally as part of cancer treatment to control malignant cells. Radiation therapy is commonly applied to the cancerous tumor because of its ability to control cell growth. Ionizing radiation works by damaging the DNA of exposed tissue, furthermore, it is believed that cancerous cells may be more susceptible to death by this process as many have turned off their DNA repair machinery during the process of becoming cancerous. To spare normal tissues (such as skin or organs which radiation must pass through in order to treat the tumor), shaped radiation beams are aimed from several angles of exposure to intersect at the tumor, providing a much larger absorbed dose there than in the surrounding, healthy tissue. Besides the tumor itself, the radiation fields may also include the draining lymph nodes if they are clinically or radiologically involved with tumor, or if there is thought to be a risk of subclinical malignant spread. It is necessary to include a margin of normal tissue around the tumor to allow for uncertainties in daily set-up and internal tumor motion. These uncertainties can be caused by internal movement (for example, respiration and bladder filling) and movement of external skin marks relative to the tumor position.

In biology, a “clonotype” is a collection of samples that are substantially similar and/or identical (i.e. clonal).

“Formalin-fixed, paraffin-embedded” (“FFPE”) tissues are a common way to preserve tissue samples.

An “adjuvant” is a pharmacological or immunological agent that modifies the effect of other agents, such as a drug or vaccine. They are often included in vaccines to enhance the recipient's immune response to a supplied antigen, while keeping the injected foreign material to a minimum Adjuvants in immunology are often used to modify or augment the effects of a vaccine by stimulating the immune system to respond to the vaccine more vigorously, and thus providing increased immunity to a particular disease. Adjuvants accomplish this task by mimicking specific sets of evolutionarily conserved molecules, so called PAMPs, which include liposomes, lipopolysaccharide (LPS), molecular cages for antigen, components of bacterial cell walls, and endocytosed nucleic acids such as double-stranded RNA (dsRNA), single-stranded DNA (ssDNA), and unmethylated CpG dinucleotide-containing DNA. Because immune systems have evolved to recognize these specific antigenic moieties, the presence of an adjuvant in conjunction with the vaccine can greatly increase the innate immune response to the antigen by augmenting the activities of dendritic cells (DCs), lymphocytes, and macrophages by mimicking a natural infection. Furthermore, because adjuvants are attenuated beyond any function of virulence, they pose little or no independent threat to a host organism.

One embodiment provides a method for identifying the DNA or RNA sequences of lymphocyte receptors expressed by lymphocytes that are present in increased numbers in a particular patient after a medical procedure; the method comprising: (i) drawing blood at one or more times from a cancer patient prior to a medical procedure (ii) carrying out a medical procedure; (iii) drawing blood from the patient at one or more times following the medical procedure; (iv) purifying a lymphocyte subpopulation, isolating DNA, amplifying (if necessary) and sequencing the genetic loci of receptors; and (v) identifying lymphocytes and/or lymphocyte receptor sequences that have expanded in number following the medical procedure.

Another embodiment provides a method wherein the analysis of lymphocyte receptor repertoire sequences, from patient samples obtained both before and after a medical procedure, enables the production of autologous genetically engineered T cells having transgenic T cell receptors which are specific to the patient's cancer.

Another embodiment provides a method wherein the analysis of lymphocyte receptor repertoire sequences, from patient samples obtained both before and after a medical procedure, enables the production of autologous genetically engineered T cells having chimeric antigen receptors which are specific to the patient's cancer.

Another embodiment provides a method wherein autologous engineered T cells, having transgenic T cell receptors (TCRs) or chimeric antigen receptors (CARs), are shown to be efficacious in-vitro in mounting a cytotoxic and/or therapeutic immune response to the presence of tumor tissue or lysate.

Another embodiment provides a method wherein autologous engineered T cells, having transgenic T cell receptors (TCRs) or chimeric antigen receptors (CARs), are shown to be efficacious in-vivo in affecting clinical endpoints such as overall survival, progression free survival, or tumor regression.

Another embodiment provides a method wherein autologous engineered T cells, having transgenic T cell receptors (TCRs) or chimeric antigen receptors (CARs), are shown to be efficacious in-vivo in affecting surrogate endpoints such as longitudinal measurements of biomarker levels or circulating tumor cells.

Another embodiment provides a method wherein the patient is selected based on the severity of cancer (for example, a Gleason score in the context of prostate cancer), patient treatment status (for example, androgen deprivation therapy status in the context of prostate cancer), or other clinical status.

Another embodiment provides a method wherein the medical procedure, comprises one or more surgical procedures, nonsurgical interventions or pharmaceutical treatments.

Another embodiment provides a method wherein the cancer is prostate cancer and the medical procedure comprises one or more of cryosurgery, radical prostatectomy, prostate biopsy, radiation therapy, brachytherapy, CyberKnife™ procedures, electroporation, high frequency ultrasound (HIFU), photodynamic therapy, prostate laser surgery, androgen deprivation therapy, and chemotherapy.

Another embodiment provides a method wherein the intervention leads to an immunogenic response.

Another embodiment provides a method wherein the intervention results in a change in the population or repertoire of lymphocytes.

Another embodiment provides a method wherein the lymphocytes include T-cells displaying various T Cell Receptors (TCRs).

Another embodiment provides a method wherein the lymphocytes include B-cells displaying various B Cell Receptors (BCRs).

Another embodiment provides a method wherein the lymphocytes include cells displaying various cell surface molecules (e.g. cluster of differentiation or cluster of designation molecules) which are used for immunophenotyping of lymphocytes.

Another embodiment provides a method of treatment of cancer comprising producing and providing the patient with autologous engineered T cells incorporating receptor sequences identified by the method of claim 1.

Another embodiment provides a method further comprising selecting clonotypes as highly expanded if their frequency (in the measured repertoire) is 0.5% or greater.

Another embodiment provides a method further comprising selecting a clonotype that is absent or not highly expanded prior to a medical procedure, but which is highly expanded after a medical procedure as a tumor associated clonotype.

Another embodiment provides a method further comprising selecting a clonotype as a tumor specific clonotype if it is highly expanded both before and after a medical procedure, but has a frequency that increases from before to after a medical procedure, wherein the increase is statistically significant using an appropriate multiple hypothesis testing statistical method to stringently limit the false discovery rate.

Another embodiment provides a method wherein the sequences of two chains comprising a lymphocyte receptor (e.g. alpha and beta TCR, gamma and delta TCR, or heavy chain and light chain BCR) are paired.

Another embodiment provides a method wherein chain pairing is carried out in silico by computer methods.

Another embodiment provides a method wherein chain pairing involves immunology gene alignment software.

Another embodiment provides a method wherein the software is selected from IMGT, JOINSOLVER, VDJSolver, SoDA, iHMMune-align, or other similar tools.

Another embodiment provides a method wherein chain pairing involves using VDJ antibodies.

Another embodiment provides a method comprising obtaining antibodies for the identified gene segments and using the antibodies to purify a subset of cells which express that gene segment in their (surface) receptors (e.g. using FACS, or immunomagnetic selection with microbeads).

Another embodiment provides a method further comprising sequencing a subset of cells which have been purified for the desired gene segments.

Another embodiment provides a method wherein chain pairing is carried out using multi-well sequencing.

Another embodiment provides a method further comprising genetic engineering of autologous T-cells to display the TCR of the induced clonotype(s).

Another embodiment provides a method wherein a T-cell is engineered to display a functional TCR.

Another embodiment provides a method wherein a chimeric cell is engineered in which a T-cell displays an alternative type of receptor such as a B-cell receptor.

Another embodiment provides a method for obtaining tumor tissue from the patient.

Another embodiment provides a method further comprising an in-vitro assay.

Another embodiment provides a method wherein, the engineered T-cells are incubated with tumor tissue and/or lysate.

Another embodiment provides a method wherein one or more effects are measured during the incubation such as cytokine concentration, cell proliferation, and the like. In some embodiments, the effects of various adjuvants are quantified.

Another embodiment provides a method wherein engineered T cells are infused or provided in-vivo to treat cancer.

Another embodiment provides a method wherein the clinical benefit of treatment with autologous engineered T cells is measured in terms of clinical endpoints such as overall survival, progression free survival, or tumor regression.

Another embodiment provides a method wherein the clinical benefit of treatment with autologous engineered T cells is measured in terms of surrogate endpoints such as longitudinal measurements of biomarker levels or circulating tumor cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Autologous Engineered T Cells for the Treatment of Prostate Cancer (Prophetic Example)

We recruit patients under an institutional review board approved protocol for human subjects and obtain written informed consent.

Patient eligibility criteria are as follows: histologically confirmed adenocarcinoma of the prostate. We obtain a clinically diverse set of patients in order to analyze the relationship between the appearance of induced clonotypes to the clinical characteristics of the patient. This diversity includes patients with localized and non-localized disease. This diversity includes patients with or without prior history of treatment (e.g., hormone deprivation treatment). This diversity includes a variety of stages of the disease.

We obtain a complete medical history at the time of intervention (where possible), including PSA (Prostate Specific Antigen) measurements, tumor grading/scoring information such as Gleason scores, etc.

Similarly, we obtain the follow-up clinical information after the procedure, including longitudinal PSA. We analyze the relationship between the appearance of induced clonotypes and the course of disease and response to treatment following the intervention.

Medical procedures include the following: cryosurgery, radical prostatectomy, prostate biopsy, radiation therapy, brachytherapy, CyberKnife™ procedures, electroporation, high frequency ultrasound (HIFU), photodynamic therapy, prostate laser surgery, androgen deprivation therapy, chemotherapy, and others. Note that prostate biopsies are typically considered to be diagnostic procedures rather than therapeutic procedures, but it is known that circulating tumor cells may increase in the days after a biopsy, so in this context we include it in our list of medical procedures of interest. Note that radical prostatectomies induce an immune response, even with the removal of tumor tissue, due to tumor tissue shedding caused by the surgery.

We obtain as complete a description of the procedure as possible. For cryosurgery, this includes the thermal timecourse used (which includes number of freezings, speed of freezings, etc.), the spatial extent and direction of the freezings, and other clinical parameters of the intervention, including adjuvants and/or medications. We obtain similar information for each intervention, in order to analyze the relationship between the clinical parameters of the intervention and the appearance of induced clonotypes.

We obtain a minimum of two blood samples—one prior to the medical procedure and one after the medical procedure (e.g. 7 days afterwards). Where possible, we obtain one or more additional samples at later time points. Where possible, we obtain more than one prior blood sample. These blood samples are peripheral blood, obtained by venipuncture. Blood from other locations is typically much less readily available. However, if blood from additional locations is available, at the discretion of the treating physician, we obtain additional blood samples. These other locations may potentially include tumor infiltrating lymphocytes from the tumor tissue, blood cells from lymph nodes (e.g. if a lymph node biopsy is done), or from other organs from which blood cells are available.

Sample collection is as follows: each sample consists of 10 mL of blood. We isolate peripheral blood mononuclear cells by flotation on Ficoll-Hypaque. We enrich multiple lymphocyte subsets from freshly isolated peripheral blood mononuclear cells by immunomagnetic selection with microbeads (e.g. Miltenyi CD8+ kit) or other separation methods such as FACS (Fluorescence-Activated Cell Sorting). We isolate one or more of the following subsets: B cells, CD8+ T cells, CD4+ T cells, CD4 Th1 cells, CD4 Th2 cells, CD4 Th17 cells, Treg cells (nTreg, iTreg, Th3, Tr1), NKT cells, and/or gamma-delta T cells. Depending on blood volumes, we may separate into subsets based on naïve, effector, or memory subsets.

We extract total genomic DNA from sorted cells using the QIAamp DNA Blood Mini kit (Qiagen) or similar kits, or commercial services providing DNA extraction or isolation. We prepare and ship DNA as per sequencing vendor instructions: at a concentration of approximately 50 ng/uL, with an A 260/280 ratio of at least 1.8, and shipped with dry ice using a vendor supplied shipping container.

Note that this procedure results in pooled genomic DNA. Alternative methods such as high throughput microdroplet based analysis (RainDance, inc.) provide single cell sequencing rather than pooled sequencing. With pooled sequencing, we carry out an additional analysis step for pairing receptor chains, described in detail below. However, if single cell sequencing is commercially available, we may extract genomic DNA using single cell technology as per vendor instructions, rather than pooling the genomic DNA, as described above.

From CDR3 sequence data provided by the vendor, we identify induced clonotypes, as follows. We define an induced clonotype as a clonotype whose change in frequency from a prior sample to a post-intervention sample is above a defined threshold. We define the threshold as 0.5%, a conservative frequency which is used to define a highly expanded clone, and which is supported by vendor reproducibility data. The analysis is also repeated with higher and lower thresholds (down to 0.1%). We rank and characterize clonotypes as weakly or strongly emergent based on their percentage increase in frequency. For example, a clonotype which had a frequency of 0.1% prior to intervention and 0.9% after intervention has an increase in frequency of 0.8%; since this is greater than the minimum increase of 0.5%, we characterize this clonotype as induced.

With multiple patient samples, we analyze the data for the presence of public clonotypes—identical (or similar) sequences which arise in parallel in multiple patients. These clonotypes represent a valuable grouping/segmentation of the patients. We analyze the clonotype groupings, if any, in relation to clinical outcomes. For in vivo and in vitro treatments, described below, we also analyze whether particular public clonotypes have favorable or unfavorable responses to in vitro or in vivo treatment.

We construct an autologous engineered T or B cell using the induced clonotype information that we identify as described above. This engineered T or B cell is the basis of an immunotherapy which we test in-vitro and eventually use therapeutically in-vivo.

With induced or expanded clonotypes, we further characterize the lymphocyte receptors as following. Several receptors consist of two chains, which are paired in vivo. For example, in T cells, a receptor may consist of an alpha and a beta chain; a different receptor may consist of a gamma and a delta chain. In B cells, the two chains are the heavy chain and the light chain. In the following explanation, for convenience, we refer to alpha and beta chains, but a similar strategy is used for pairing heavy chains and light chains, or gamma chains and delta chains.

We sequence these chains as described above. Note that if single cell or single clonotype sequencing is available, this pairing step is not necessary—if cells are sequenced individually, the chain pairing is known without further effort. If pooled sequencing is used, then we have a list of induced alpha chains, and a list of induced beta chains, (or heavy chains and light chains, etc), and we now pair the chains.

In order to pair the chains, we benefit from the fact that these chains are made in vivo via VDJ recombination. Furthermore, V and J gene segment specific antibodies are readily (commercially) available.

Therefore, we start with one chain—for example, the beta chain. We identify an induced clonotype, and from its sequence, we identify its V and J gene segments, as follows.

We can annotate the induced clonotype's gene segments from its sequence using specialized, publicly available immunology gene alignment software from IMGT (International IMmunoGeneTics information system, www.imgt.org, V-Quest, JunctionAnalysis, etc), JOINSOLVER, VDJSolver, SoDA, iHMMune-align, or other similar tools.

We obtain antibodies for the identified segments. We use the antibodies to purify a subset of cells which express that gene segment in their (surface) receptors (e.g. using FACS, or immunomagnetic selection with microbeads). Finally, using this subset of cells which have been purified for the desired gene segments, we sequence again, as describe above, or possibly in a less expensive, more low-throughput manner. If necessary, we sequence this subset more deeply (i.e. at a higher resolution) than previously. In this new data set, there will be far fewer induced clonotypes, greatly easing the task of chain pairing. Depending on the gene segments, there may be only one induced alpha chain and one induced beta chain for example.

Alternatively, (e.g. if the above method is inconclusive), we can isolate our gene segment purified cells (or even unpurified cells) into a microwell plate, where each microwell has a very low number of cells. We can amplify and sequence the cells in each well individually, which provides another means to pair the chains of interest by sequencing on a single cell basis (or single clonotype basis, if we treat the cells (e.g. with adjuvants) to proliferate in the microwells prior to sequencing them.)

Once we pair the alpha/beta, gamma/delta, or heavy/light chains of the induced clonotypes, we engineer an autologous B or T cell which expresses a receptor corresponding to the induced clonotype. This receptor may be a T cell receptor on a T cell surface, or a chimeric antibody receptor. The chimeric antibody receptor may be comprised of a single chain variable fragment (scFv) on the T cell surface. The chimeric antibody may be enhanced by the presence of costimulatory endodomains.

The following procedure describes the engineering of a T cell receptor on a T cell surface. The tools for this process are commercially available and a great deal of literature describes this process. In particular, recently published work with T cells and CARs (chimeric antigen receptors) provides rich guidance regarding methods to enhance the potency of the engineered T cells (in particular, third generation CARs).

When engineering a T cell which expresses a desired (induced) T cell receptor sequence, we prepare by acquiring a suitable lentiviral or other retroviral vector. A number of commercial vendors can construct customized lentiviral vectors, and a number of kits for lentiviral transduction are available. In the alternative, it may be beneficial to use other kinds of vectors. In summary, we obtain commercially engineered lentiviral particles in which the desired (induced) TCR or CAR sequences have been introduced. In addition, we acquire a suitable population of T cells from the patient via leukapheresis, and maintain them ex vivo.

Following vendor instructions, we then incubate the T cell population with the lentivirus. Commonly, cytokines such as IL-2 or IL-7 are used to facilitate this process; in this case, we follow vendor instructions.

We confirm the success of the transduction, and the expression of the engineered T cells in multiple ways. First, we use VDJ gene segment specific antibodies, as described previously. Second, we sequence the engineered cells, as described previously. We use additional verification methods as appropriate.

We test our engineered T cells in-vitro by incubating them with tumor tissue lysate and also with various combinations of adjuvants such as GM-CSF, IL-2, and others. We measure efficacy using assays for cytokines (e.g. IFN-gamma) and T cell proliferation. These assays are commercially available, and we follow vendor instructions.

Finally, for our in vivo study, we follow FDA guidance and Good Manufacturing Procedures to produce engineered T cells for in-vivo use. We adhere to extensive regulatory guidance in developing the necessary procedures. In vivo, efficacy is measured through surrogate endpoints (e.g. longitudinal PSA, circulating tumor cells) and clinical endpoints (e.g. overall survival, progression free survival, tumor regression).

What is claimed is:

1. A method for identifying DNA or RNA sequences of lymphocyte receptors that are present in greater numbers of lymphocytes after a medical procedure; the method comprising:
(i) drawing pre-procedure blood from a cancer patient;
(ii) carrying out a medical procedure on the patient;
(iii) drawing blood from the patient at one or more times following the medical procedure;
(iv) purifying lymphocytes from any one or more of the pre-procedure blood draw(s), and/or the post-procedure blood draw(s); isolating DNA, amplifying (if necessary) and sequencing the DNA; and
(v) identifying lymphocytes and/or lymphocyte receptor sequences that have expanded following the procedure.

2. The method of claim 1 wherein the patient is selected based on the severity of cancer (Gleason Score for example) and/or patient treatment status.

3. The method of claim 1 wherein the procedure comprises one or more surgical procedure or procedure, nonsurgical procedure or procedure, or exposure to a drug.

4. The method of claim 3 wherein the cancer is prostate cancer and the medical procedure comprises one or more of cryosurgery, radical prostatectomy, prostate biopsy, radiation therapy, brachytherapy, CyberKnife™ procedures, electroporation, high frequency ultrasound (HIFU), photodynamic therapy, prostate laser surgery, androgen deprivation therapy, and chemotherapy.

5. The method of claim 1 wherein the procedure results in a change in the population lymphocytes.

6. A method of treatment of cancer comprising inducing in a patient an immunologic response incorporating clonetypes identified by the method of claim 1.

7. The method of claim 6 further comprising selecting clonotypes as highly expanded if their frequency (in the measured repertoire) is 0.5% or greater.

8. The method of claim 6 further comprising selecting a clonotype that is absent or not highly expanded prior to cryosurgery, but which is highly expanded after cryosurgery as a tumor associated clonotype.

9. The method of claim 6 further comprising selecting a clonotype as a tumor specific clonotype if it is highly expanded both before and after a medical procedure, but has a frequency that increases from before to after the procedure, wherein the increase is statistically significant using an appropriate multiple hypothesis testing statistical method to stringently limit the false discovery rate.

10. The method of claim 1 further comprising extracting tissue from the patient for use in an in vitro assay of autologous engineered T cells.

11. The method of claim 1 wherein the procedure comprises receptor chain pairing.

12. The method of claim 11 wherein chain pairing involves immunology gene alignment software.

13. The method of claim 12 wherein the software is selected from IMGT, JOINSOLVER, VDJSolver, SoDA, iHMMune-align, or other similar tools.

14. The method of claim 11 wherein chain pairing involves using VDJ antibodies.

15. The method of claim 14 comprising obtaining antibodies for the identified segments and use the antibodies to purify a subset of cells which express that gene segment in their (surface) receptors (e.g. using FACS, or immunomagnetic selection with microbeads).

16. The method of claim 15 further comprising sequencing a subset of cells which have been purified for the desired gene segments.

17. The method of claim 11 wherein chain pairing is carried out using multi-well sequencing or single cell sequencing.

18. The method of claim 10 further comprising genetic engineering of autologous T-cells, acquired by leukapheresis, to display the TCR or CAR of the induced clonaltype(s).

19. The method of claim 18 wherein a T-cell is engineered to display a functional TCR.

20. The method of claim 19 wherein a chimeric cell is engineered in which a T-cell displays an alternative type of receptor such as a chimeric antigen receptor.
